(19) United States
(12) Patent Application Publication
(10) Pub. No.: US 2010/0144597 A1
(43) Pub. Date: Jun. 10, 2010

(54) Novel combinatorial approaches to enhancing oxygen transport to tissues

(76) Inventors: Kevin R. Ward, Glen Allen, VA (US); Bruce Spiess, Manakin-Sabot, VA (US)

Correspondence Address: COOPER & DUNHAM, LLP, 30 Rockefeller Plaza, 20th Floor, NEW YORK, NY, 10112, US

(21) Appl. No.: 12/653,343

(22) Filed: Dec. 10, 2009

Related U.S. Application Data
(60) Provisional application No. 61/201,492, filed on Dec. 10, 2008.

(51) Int. Cl. A61K 38/42; A61K 31/025; A61K 31/202; A61K 31/519; A61P 43/00

(52) U.S. Cl. 514/6; 514/747; 514/560; 514/252.16

(57) ABSTRACT

The subject application provides for a method for enhancing oxygen delivery to a tissue in a subject comprising administering to the subject a composition comprising two or more compounds selected from the group consisting of a hemoglobin based oxygen carrier, a nonhemoglobin based oxygen carrier, a rheologic modulator, a diffusion changing compound, a volume expander, and a vasomodulator. The subject application also provides for a method for enhancing oxygen delivery to a tissue in a subject comprising administering to the subject a composition comprising a perfluorocarbon and one or more compounds selected from the group consisting of a hemoglobin based oxygen carrier, a nonhemoglobin based oxygen carrier, a rheologic modulator, a diffusion changing compound, a volume expander, and a vasomodulator.

This application claims the benefit of U.S. Provisional Application No. 61/201,492, filed Dec. 10, 2008, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Despite decades of research, medicine has yet to produce a readily usable oxygen carrying compound capable of meeting diverse medical needs. A major barrier to its development has been confusion as to the when, why, and where to use such a compound as well as a misdirected understanding of the role of transfusion. Past research has focused on developing a sole replacement for the packed unit of red blood cells. This approach, however, is inadequate for meeting diverse medical needs since there are numerous conditions which would benefit from enhanced oxygen delivery but for which the infused red blood cells would not achieve the desired result.

There are three basic blood substitute approaches to enhancing oxygen delivery to tissues. The existing methods include single mediator oxygen therapeutics utilizing hemoglobin based oxygen carriers (HBOCs), nonhemoglobin based oxygen carriers (e.g., perfluorocarbons), and diffusion-rheologic compounds such as the rheologic changing material Drag-Reducing Polymer (DRP) and the diffusion changing compound crocetin. Each of these methods is capable of enhancing oxygen delivery to tissues in a unique manner.

In addition to the use of oxygen carrying compounds, other methods for the enhancement of oxygen delivery to tissues include simple volume expansion via use of a simple volume expander (e.g., hypertonic saline), manipulation of the vasculature to cause microcirculatory vasodilation, which can include the use of vasomodulators (e.g., sildenafil), methods affecting tissues to consume more oxygen, and even the intravascular production of oxygen.

Despite the existence of varying techniques for enhancing oxygen delivery to tissues, not all conditions in need for greater local or systemic tissue oxygenation can be treated by one of these techniques alone. Pathologies resulting in reduction of oxygen delivery and utilization of tissues resulting in death or tissue destruction are complex. Multiple mechanisms are in play making a simple single compound solution to the problem untenable. This is likely the major reason for the lack of significant improvement in outcome which has resulted from oxygen therapeutics. Methods for treatment of these conditions are needed.

### SUMMARY OF THE INVENTION

The subject application provides for a method for enhancing oxygen delivery to a tissue in a subject comprising administering to the subject a composition comprising two or more compounds selected from the group consisting of a hemoglobin based oxygen carrier, a nonhemoglobin based oxygen carrier, a rheologic modulator, a diffusion changing compound, a volume expander, and a vasomodulator.

The subject application also provides for a method for enhancing oxygen delivery to a tissue in a subject comprising administering to the subject a composition comprising a perfluorocarbon and one or more compounds selected from the group consisting of a hemoglobin based oxygen carrier, a nonhemoglobin based oxygen carrier, a rheologic modulator, a diffusion changing compound, a volume expander, and a vasomodulator.

### DETAILED DESCRIPTION OF THE INVENTION

The subject application provides for a method for enhancing oxygen delivery to a tissue in a subject comprising administering to the subject a composition comprising two or more compounds selected from the group consisting of a hemoglobin based oxygen carrier, a nonhemoglobin based oxygen carrier, a rheologic modulator, a diffusion changing compound, a volume expander, and a vasomodulator.

In one embodiment, the nonhemoglobin based oxygen carrier is a perfluorocarbon. In another embodiment, the perfluorocarbon is perfluoro(tert-butylcyclohexane) (“FtBu”).

In one embodiment, the rheologic modulator is a drag-reducing polymer. In another embodiment, the diffusion changing compound is crocetin. In another embodiment, the volume expander is hypertonic saline solution. In yet another embodiment, the vasomodulator is sildenafil.

In one embodiment, the subject is affected by a pathological condition. In another embodiment, the pathological condition is a wound, a burn, traumatic shock, traumatic brain injury, organ ischemia, stroke, spinal cord injury, myocardial infarction, acute limb ischemia, mesenteric ischemia, isolated organ injury, congestive heart failure, adult respiratory distress syndrome, birth asphyxia, vasoocclusive crisis, hemorrhage, subarachnoid hemorrhage, systemic states of hypoxia, cardiac arrest, sepsis, decompression illness or acute respiratory disease (ARDS).

The subject application also provides for a method for enhancing oxygen delivery to a tissue in a subject comprising administering to the subject a composition comprising a perfluorocarbon and one or more compounds selected from the group consisting of a hemoglobin based oxygen carrier, a nonhemoglobin based oxygen carrier, a rheologic modulator, a diffusion changing compound, a volume expander, and a vasomodulator. In one embodiment, the perfluorocarbon is perfluoro(tert-butylcyclohexane).

In one embodiment, the rheologic modulator is a drag-reducing polymer. In another embodiment, the diffusion changing compound is crocetin. In another embodiment, the volume expander is hypertonic saline solution. In yet another embodiment, the vasomodulator is sildenafil.

In one embodiment, the subject is affected by a pathological condition. In another embodiment, the pathological condition is a wound, a burn, traumatic shock, traumatic brain injury, organ ischemia, stroke, spinal cord injury, myocardial infarction, acute limb ischemia, mesenteric ischemia, isolated organ injury, congestive heart failure, adult respiratory distress syndrome, birth asphyxia, vasoocclusive crisis, hemorrhage, subarachnoid hemorrhage, systemic states of hypoxia, cardiac arrest, sepsis, decompression illness or acute respiratory disease (ARDS). In one embodiment, the subject is a mammal. In another embodiment, the mammal is a human.

Terms

As used herein, and unless stated otherwise, each of the following terms shall have the definition set forth below.

“Administering to the subject” means the giving of, dispensing of, or application of medicines, drugs, or remedies to a subject to relieve or cure a pathological condition. As used herein, “administering” an agent, for example a perfluorocarbon (“PFC”), may be performed using any of the various methods or delivery systems well known to those skilled in the art. The administering can be performed, for example, intravenously (including intra-arterially), intrathecally, parenterally, topically, or by ventilation.

“Antibacterial agent” means a bactericidal compound such as silver nitrate solution, mafenide acetate, or silver sulfadiazine, or an antibiotic.

“Biologically active agent” means a substance which has a beneficial or adverse effect on living matters.

“Effective” as in an amount effective to achieve an end means the quantity of a component that is sufficient to yield a desired therapeutic response without undue adverse side effects (such as toxicity, irritation, or allergic response) commensurate with a reasonable benefit/risk ratio when used in the manner of this disclosure. For example, an amount effective to promote wound healing without causing undue adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

A “diffusion changing compound” is a compound which modulates the diffusion of matter.

“HBOCs”, or hemoglobin based oxygen carriers, are modified hemoglobins used as blood substitute products. HBOCs generally comprise a homogenous aqueous solution of chemically-modified hemoglobin, essentially free from other red cell residue (stroma). Although stroma-free hemoglobin (SFH) from humans is the most common raw material for preparing a HBOC, other sources of hemoglobin have also been used. For example, hemoglobin can be obtained or derived from animal blood (e.g., bovine or porcine hemoglobin) or from bacteria or yeast or transgenic animals or plants molecularly altered to produce a desired hemoglobin product. A “nonhemoglobin based oxygen carrier” means an oxygen carrying compound not derived from hemoglobin. One exemplary embodiment of a nonhemoglobin based oxygen carrier is perfluorcarbon.

“Oxygenated perfluorocarbon” is a perfluorocarbon which is carrying oxygen at, for example, saturation or sub-saturation levels.

“Pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

“Pharmaceutically active compound” means the compound or compounds that are the active ingredients in a pharmaceutical formulation.

A “rheologic modulator” is a compound which modulates the flow of matter.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention.

A “volume expander” is a compound which expands the volume of matter.

A “vasomodulator” is a compound which modulates blood vessels. Vasomodulators include vasodilators. Vasodilators are compounds which widens blood vessels. When vessels dilate, the flow of blood is increased.

Perfluorocarbons

Perfluorocarbons (PFCs) possess the ability to dissolve large quantities of many gases at concentrations much larger than water, saline and plasma. In addition, PFCs enhance the ability of these gases to diffuse across distances. Thus, PFCs can be a convenient and inexpensive means to deliver high levels of oxygen or other therapeutic gases to wounds and other organ systems.

Being that the PFCs are slightly lipophilic at body temperature and would help in the transport of oxygen into and removal of carbon dioxide from the skin tissue, PFCs can accelerate the healing process of a wound in a tissue. A preferred PFC, Perfluoro(tert-butylcyclohexane), is only slightly lipophilic at body temperature and not lipophilic at room temperature.

PFCs that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of gases, both as emulsions for intravenous use and as neat liquids for liquid ventilation applications. Use of perfluorochemicals in biological gas exchange, for example as a blood substitute, or for intra-pulmonary or liquid ventilation applications, is described in U.S. Pat. No. 5,674,913, issued Oct. 7, 1997 to Clark, Jr., and in U.S. Pat. No. 5,840,767, issued Nov. 24, 1998 to Clark, Jr. et al., which are incorporated herein by reference.

PFCs include perfluoro(tert-butylcyclohexane) (C₁₀F₂₀) which is available, for example, as Oxycyte™ from Oxygen Biotherapeutics, Inc., Costa Mesa, Calif. In an embodiment, the perfluoro(tert-butylcyclohexane) has the following structure:

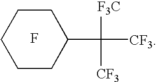

Oxycyte™ is a perfluorocarbon emulsion oxygen carrier. The active ingredient in Oxycyte™, perfluoro(tert-butylcyclohexane) (C₁₀F₂₀, MW-500), also known as F-tert-butylcyclohexane or “FtBu”, is a saturated alicyclic PFC. Perfluoro(tert-butylcyclohexane) is a colorless, completely inert, non-water soluble, non-lipophilic molecule, which is twice as dense as water, and boils at 147° C.

Physical properties of Perfluoro(tert-butylcyclohexane) are as follows:

Molecular Formula: C₁₀F₂₀

Molecular Weight (g/mol): 500.08

Physical State@Room Temperature: Liquid

Density (g/mL): 1.97

Boiling Point (° C.): 147

Vapor Pressure (mmHg)@25° C.: 3.8

Vapor Pressure (mmHg)@37° C.: 4.4

Kinematic Viscosity (cP): 5.378

Refractive Index@20° C.: 1.3098

Calculated Dipole Moment (Debye): 0.287

Calculated Surface Tension (dyne/cm): 14.4

Perfluoro(tert-butylcyclohexane) carries about 43 mL of oxygen per 100 mL of PFC, and 196 mL of CO₂ per 100 mL of PFC.

Use of perfluoro(tert-butylcyclohexane) for liquid ventilation and artificial blood applications is described in U.S. Pat. No. 6,167,887, issued Jan. 2, 2001 to Clark, et al., which is incorporated herein by reference.

As formulated and manufactured, Oxycyte™ is a sterile, non-pyrogenic emulsion consisting of submicron particles (medium diameter 150-300 nanometers) of perfluoro(tert-butylcyclohexane) in an aqueous medium that is isotonic and mildly buffered to a neutral pH range. To be physiologically compatible the PFC in Oxycyte™ is emulsified with egg-yolk phospholipids. A representative composition of the 60% w/v Oxycyte™ emulsion is shown in Table 1.

TABLE 1
Representative Composition of Oxycyte ™ 60% w/v

| Component | mg/mL |
|---|---|
| Perfluoro(tert-butylcyclohexane) | 600.0 |
| Sodium Phosphate monobasic | 0.52 |
| Monohydrate | |
| Sodium Phosphate Dibasic Heptahydrate | 3.55 |
| Glycerin | 12.7 |
| Calcium Disodium Edetate Dihydrate | 0.2 |
| Egg Yolk Phospholipid | 40.0 |
| Vitamin E (dl-alpha-tocopherol) | 0.05 |
| Water for Injection (WFI) | 638.7 |

In the body the PFC emulsion is capable of uploading and unloading oxygen and CO₂ more efficiently than blood, and this process is concentration-gradient mediated (Henry's Law). Because the median size of the PFC droplets is approximately 40-50 times smaller than an erythrocyte, Oxycyte™ is able to oxygenate tissues with narrowed capillaries, as occurs in brain contusions. Oxycyte™ remains in the circulation for 20 to 24 hours after a single 30 minute rapid infusion of 3 mL/kg. PFCs are eliminated from the blood when macrophages scavenge the lipid particles. This is quite similar to how Intralipid® is transported from the blood stream. PFCs are deposited in the liver and spleen. The lipid emulsion is slowly broken down liberating PFC to be carried back to the lungs on various proteins and lipids wherein they are breathed out as a colorless, odorless and tasteless vapor. In non-human primates, the half-life of PFC in the liver and spleen was found to be dose related; at a dose of 1.8 g/kg (3 mL/kg), the half-life is approximately 12 days.

Injectable PFC emulsions can comprise excipients such as solubility-altering agents (e.g., ethanol, propylene glycol and sucrose) and polymers (e.g., polycaprylactones and PLGA's) as well as pharmaceutically active compounds. The PFC emulsions may also comprise other pharmaceutically acceptable carriers suitable for intravenous or intrathecal administration.

The perfluorocarbon emulsions of the methods and uses of the invention include perfluorocarbon-in-water emulsions comprising a continuous aqueous phase and a discontinuous perfluorocarbon phase. The emulsions typically include emulsifiers, buffers, osmotic agents, and electrolytes. The perfluorocarbons are present in the emulsion from about 5% to 130% w/v. Embodiments include at least about 40%, 45%, 50%, 55%, 60%, 65%, 70%, 75%, 80% and 85% w/v. A 60% w/v perfluoro(tert-butylcyclohexane) emulsion may be used as the perfluorocarbon emulsion in one embodiment. Embodiments also include an egg yolk phospholipid emulsion buffered in an isotonic medium wherein the perfluorocarbon is present in the emulsion from about 5% to 130% w/v. Embodiments include at least about 40%, 45%, 50%, 55%, 60%, 65%, 70%, 75%, 80% and 85% w/v. A 60% w/v perfluoro(tert-butylcyclohexane) emulsion may be used as the perfluorocarbon emulsion in one embodiment of an egg yolk phospholipid emulsion buffered in an isotonic medium.

The composition employed in the methods described herein may comprise a pharmaceutically acceptable additive.

Combinatorial Approach

Because the existing tissue oxygenation approaches are essentially exclusive of each other, they can be mixed and matched so as to not be fundamentally limited by the underlying pathology. The present invention provides for a novel combinatorial approach utilizing unique combinations of products to restore tissue oxygenation based on the cause of tissue oxygenation reduction in that particular pathology. The present invention combines unique agents to enhance the delivery and utilization of oxygen to tissues. The various configurations of agents provide clinicians unique options to enhance tissue oxygenation depending on the pathologic situation at hand. Such combinatorial approach to tissue oxygenation was not previously disclosed.

The combinatorial approach to enhancing oxygen delivery to tissues is useful for treating any and all varieties of conditions which would be improved through the enhancement of oxygen delivery to tissues. These may range from single organ ischemia such as myocardial infarction, stroke, traumatic brain injury, spinal cord injury, acute limb ischemia, mesenteric ischemia, etc. to systemic states of hypoxia such as cardiac arrest, traumatic shock, sepsis, decompression illness, burns, vasoocclusive crisis, hemorrhage, acute respiratory disease, etc. The combinatorial approach is also contemplated for use in organ preservation and in wound care.

One embodiment of the present invention provides for a method for enhancing oxygen delivery to a tissue in a subject comprising administering to the subject a composition comprising HBOC, PFC, a hypertonic-hyperoncotic volume expander, rheologic modulator, and vasomodulator. Such an approach would be valuable to a polytrauma patient who is experiencing a combination of traumatic shock and traumatic brain injury. The combination above would 1) increase oxygen carrying capacity, 2) increase diffusion of oxygen, 3) reduce intracranial pressure via osmotic forces, 4) enhance circulatory volume by hyperoncotic movement of interstitial volume into the vasculature, without reducing rheology, 5) improve microcirculatory blood flow via vasodilation, and 6) reduce inflammation via rheologic and immune modulation.

Similar benefits can also be seem for individual organ ischemia such as stroke, spinal cord injury, myocardial infarction, or any other type of isolated organ injury.

The combination solutions described above are envisioned to be first load products in acute settings. Subsequent treatments as needed would take the form of alternative combination products since repetitive dosing of component parts such as hypertonic saline and PFCs may be problematic. However, repetitive dosing of subsequent formulas such as additional HBOC with rheological properties such as DRPs (e.g., polyethylene glycol, polaxamer 188, etc.) and other diffusion enhancing substances such as crocetin would be feasible. Such approaches may also have the benefit of significantly enhancing oxygen delivery without the need for very high doses of supplemental oxygen.

Other novel enhancements may include but is not limited to the addition of metabolic and vasomodulating substances.

For example, the use of the compound sildenafil may cause microcirculatory vasodilation via its nitric oxide (NO) producing effects and enhance the ability of tissues to consume oxygen via its K+ ATP channel properties. The addition of nitric oxide precursors such as L-arginine is possible. Combining these with hypertonic saline or dextran solutions would limit the sudden onset of hypotension.

While nitrosylated hemoglobin has been used for reducing NO uptake (resulting in a relative increase in NO) by hemoglobins, and as a means to scavenge free radicals, it remains a single modulation of hemoglobin similar to PEGylation (i.e., the covalent attachment of poly(ethylene glycol) polymer chains to another molecule). Both of these types of HBOCs would benefit by combining with additional biocompatible methods to increase delivery of oxygen to tissues.

Such approaches provide clinicians a versatile tool box of oxygen therapeutics which can be tailored to the needs of the patient. One example would be a patient with Congestive Heart Failure (CHF) who is volume overloaded but in need of enhanced tissue oxygen delivery. The administration of PFC-HBOC combination composition with rheologic and vasodilatory agents would allow for significant enhancement of tissue oxygenation without aggravating the volume status of the patient.

The condition of cardiac arrest may require an alternative approach. Providing a PFC-HBOC-rheologic enhancer may maximally increase oxygen carrying capacity and delivery thus compensating for poor flow. The addition of novel intravascular oxygen production strategies like encapsulated H₂O₂ to produce oxygen at ischemic tissues would also serve to offset the profound reductions in blood flow.

Use of this combinatorial approach produce means to treat conditions not previously believed to be amenable to injectable oxygen therapeutics such as adult respiratory distress syndrome and birth asphyxia, where enhancing the delivery of available oxygen may be life saving.

Even the use of simple hyperoncotic volume expanders with rheologic and vasomodulating altering compounds would find use as potential first line treatments for conditions requiring enhanced oxygen delivery. Such conditions might include vasoocclusive crisis or subarachnoid hemorrhage. These agents may improve flow and alter inflammation.

Instead of being limited by a single mode of action of any of the previous single compound therapies, the methods provided by this invention takes advantage of the many separate mechanisms involved where the methods are mutually biocompatible. The examples described herein are not exhaustive. Inhospital and out of hospital use including combat casualty care uses are envisioned.

Alternative modes of delivery may be possible by combining existing modes for delivery. For example, combinations of intravascular and extravascular delivery can be used in the case of in severe burns and other large wounds where the jeopardized tissue may not be oxygenated via the surface and intravascular routes. Other delivery modes include intraperitoneal delivery of agents along with intravascular administration to optimize splanchnic oxygenation. Intraluminal oxygenation of the gastrointestinal tract is also envisioned.

The compositions of the present invention may comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise HBOCs and perfluorocarbons. The composition employed in the methods described herein may also comprise a pharmaceutically acceptable additive.

The compositions of the present invention may contain additional beneficial biologically active agents which further promote tissue health including but is not limited to growth factors, enzymatic debridement agents, hemostatics, and others.

The compounds of the present invention may be in a salt form. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compositions of this invention may be administered in various forms, including those detailed herein. The treatment with the compound may be a component of a combination therapy or an adjunct therapy, i.e. the subject or patient in need of the drug is treated or given another drug for the pathological condition in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed. In an embodiment, a composition is provided comprising an amount of the compound effective to treat a pathological condition as specified above and a pharmaceutical carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds also used to treat the pathological condition. The compounds may also be administered in intravenous (bolus or infusion), intraperitoneal, subcutaneous, or intramuscular form, or introduced directly, e.g. by injection, topical application, or other methods, into the tissue, all using dosage forms well known to those of ordinary skill in the pharmaceutical arts.

The compounds can be administered in admixture with suitable pharmaceutically acceptable carriers i.e., pharmaceutical diluents, extenders, excipients, or carriers suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. Examples of suitable solid carriers include lactose, sucrose, gelatin and agar. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The compounds can be administered parenterally, in sterile liquid dosage forms. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Parenteral and intravenous forms may also include minerals and other materials to make them compatible with the type of injection or delivery system chosen.

The instant compounds may also be administered via transdermal routes, using those forms of transdermal skin patches well known to those of ordinary skill in that art. To be administered in the form of a transdermal delivery system, the dosage administration will generally be continuous rather than intermittent throughout the dosage regiment.

The compositions of the present invention may also contain any of the following non-toxic auxiliary substances:

The compositions of the present invention may contain antibacterial components which are non-injurious in use, for example, thimerosal, benzalkonium chloride, methyl and propyl paraben, benzyldodecinium bromide, benzyl alcohol, or phenylethanol.

The compositions of the present invention may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The compositions of the present invention may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The compositions of the present invention may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The compositions of the present invention may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The compositions of the present invention may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

It is understood that where a parameter range is provided, all integers within that range, and tenths thereof, are also provided by the invention. For example, “25-50%” includes 25.0%, 25.1%, 25.2%, 25.3%, 25.4% etc up to 50.0%.

All combinations of the various elements are within the scope of the invention.

This invention will be better understood by reference to the Experimental Details which follow, but those skilled in the art will readily appreciate that the specific experiments detailed are only illustrative of the invention as described more fully in the claims which follow thereafter.

Experimental Details

### Example 1

A composition comprising hemoglobin based oxygen carrier, PFC, hypertonic-hyperoncotic volume expander, rheologic modulator, and vasomodulator is administered to a tissue in a subject in need thereof.

Oxygen delivery to the tissue in the subject is enhanced.

1. A method for enhancing oxygen delivery to a tissue in a subject comprising administering to the subject a composition comprising two or more compounds selected from the group consisting of a hemoglobin based oxygen carrier, a nonhemoglobin based oxygen carrier, a rheologic modulator, a diffusion changing compound, a volume expander, and a vasomodulator.

2. The method of claim 1, wherein the nonhemoglobin based oxygen carrier is a perfluorocarbon.

3. The method of claim 2, wherein the perfluorocarbon is perfluoro(tert-butylcyclohexane).

4. The method of claim 1, wherein the rheologic modulator is a drag-reducing polymer.

5. The method of claim 1, wherein the diffusion changing compound is crocetin.

6. The method of claim 1, wherein the volume expander is hypertonic saline solution.

7. The method of claim 1, wherein the vasomodulator is sildenafil.

8. The method of claim 1, wherein the subject is affected by a pathological condition.

9. The method of claim 8, wherein the pathological condition is a wound, a burn, traumatic shock, traumatic brain injury, organ ischemia, stroke, spinal cord injury, myocardial infarction, acute limb ischemia, mesenteric ischemia, isolated organ injury, congestive heart failure, adult respiratory distress syndrome, birth asphyxia, vasoocclusive crisis, hemorrhage, subarachnoid hemorrhage, systemic states of hypoxia, cardiac arrest, sepsis, decompression illness or acute respiratory disease (ARDS).

10. A method for enhancing oxygen delivery to a tissue in a subject comprising administering to the subject a composition comprising a perfluorocarbon and one or more compounds selected from the group consisting of a hemoglobin based oxygen carrier, a nonhemoglobin based oxygen carrier, a rheologic modulator, a diffusion changing compound, a volume expander, and a vasomodulator.

11. The method of claim 10, wherein the perfluorocarbon is perfluoro(tert-butylcyclohexane).

12. The method of claim 10, wherein the rheologic modulator is a drag-reducing polymer.

13. The method of claim 10, wherein the diffusion changing compound is crocetin.

14. The method of claim 10, wherein the volume expander is hypertonic saline solution.

15. The method of claim 10, wherein the vasomodulator is sildenafil.

16. The method of claim 10, wherein the subject is affected by a pathological condition.

17. The method of claim 16, wherein the pathological condition is a wound, a burn, traumatic shock, traumatic brain injury, organ ischemia, stroke, spinal cord injury, myocardial infarction, acute limb ischemia, mesenteric ischemia, isolated organ injury, congestive heart failure, adult respiratory distress syndrome, birth asphyxia, vasoocclusive crisis, hemorrhage, subarachnoid hemorrhage, systemic states of hypoxia, cardiac arrest, sepsis, decompression illness or acute respiratory disease (ARDS).

18. The method of claim 1, wherein the subject is a mammal.

19. The method of claim 18, wherein the mammal is a human.
